(19) United States
(12) Patent Application Publication
(10) Pub. No.: US 2024/0374770 A1
(43) Pub. Date: Nov. 14, 2024

(54) Medical Device Sterilizer and Magnetizer

(71) Applicant: Bard Access Systems, Inc., Salt Lake City, UT (US)

(72) Inventor: Tyler L. Durfee, Stansbury, UT (US)

(21) Appl. No.: 18/195,018

(22) Filed: May 9, 2023

(51) Int. Cl. A61L 2/10; A61B 90/98; A61L 2/26; H01F 7/02

(52) U.S. Cl. CPC A61L 2/10; A61B 90/98; A61L 2/26; H01F 7/02; A61L 2202/11; A61L 2202/121; A61L 2202/122; A61L 2202/24

(57) ABSTRACT

A system, apparatus and method are directed to a magnetizer comprising at least one magnetizing element and an irradiation source. The housing defines a cavity that communicates with an opening. By placing a medical device, e.g. a needle, through the opening and into the cavity exposes the needle to the irradiation source while being magnetized to imprint a magnetic signature. Optionally, a switching mechanism can be actuated when the needle passes through/engages the opening and actuates the irradiation source and/or magnetizing element. Advantageously, the needle can be sterilized while being magnetized. Further, the cavity can be sterilized to prevent contaminating the needle during magnetization.

### BACKGROUND

Magnetic tracking of medical devices has become an important tool for determining the location and orientation of medical devices within the body. Electro-magnetic systems require complex networks of emitters and detectors in order to track such medical devices using electro-magnetic waves, and such systems can be bulky and expensive. Tracking static magnetic fields emitted by permanent magnets or magnetized medical devices can reduce both the cost and overall size of the tracking system and the tracked medical device. Further, some tracking systems imprint a unique magnetic signature onto the magnetizable medical device to allow the static magnetic tracking system to distinguish between two or more medical devices.

The process of imprinting a magnetic signature requires placing a magnetizable medical device, e.g. a needle formed of a magnetizable material, proximate a magnetizing system that exposes the needle to a magnetic field. The magnetic field imprints a unique magnetic signature onto the needle. However, in the process of which, the sterility of the medical device can be compromised. For example, when a medical device is inserted into a magnetizer for magnetization, the medical device may contact an entrance of the magnetizer. Any exposure or contact by the medical device or the user to an unsterilized magnetization device may result in loss of sterility. Furthermore, a sterilized magnetizer may make contact with unsterilized surfaces (e.g., with an unsterile skin surface) and thereby become contaminated.

Existing magnetizer systems are often formed with a thin, deep well into which the medical device is placed (termed “dipping”), and for which cleaning may be difficult. Existing magnetizer systems are therefore generally either single use, or may utilize a separate disposable piece to be inserted first into the magnetizer prior to dipping in a needle, which can be costly and/or generate excessive waste.

What is needed therefore is a system of imprinting a magnetic signature onto a medical device while maintaining the sterility of the medical device. Disclosed herein are systems and methods to address the foregoing.

### SUMMARY

Briefly summarized, embodiments disclosed herein are directed to a magnetizer having a housing defining a top face, a bottom face and one or more side faces extending therebetween, a cavity disposed within the housing and communicating with an opening disposed in the top face, the opening and cavity configured to receive a distal portion of a medical device therein, a magnetizing element disposed in a wall of the cavity, and an irradiation source disposed in a wall of the cavity and configured to direct ultraviolet radiation into the cavity.

In some embodiments, the magnetizing element is configured to magnetize a portion of the medical device.

In some embodiments, the magnetizing element is configured to imprint a magnetic signature onto the medical device.

In some embodiments, the irradiation source is configured to emit electromagnetic radiation between the wavelength ranges of 100-280 nm.

In some embodiments, the irradiation source is configured to emit one or more of UV-A, UV-B, and UV-C radiation.

In some embodiments, the irradiation source is one of a LED bulb, a light bulb, an incandescent bulb, a fluorescent bulb, a fluorescent tube, or a low-pressure mercury-vapor gas-discharge lamp.

In some embodiments, the opening further includes a switching mechanism configured to transition one or both of the magnetizing element and the irradiation source between an activated state and a deactivated state.

In some embodiments, the opening further includes one of a door or a flap configured to mitigate radiation from the irradiation source escaping from the cavity.

In some embodiments, the medical device includes one of a needle, guidewire, or stylet that includes a magnetizable material.

In some embodiments, a proximal portion of the medical device engages the opening to, i) align a distal portion of the medical device with a central axis of the cavity, ii) align the distal portion of the medical device at a predetermined distance from the irradiations source, or iii) prevent a distal tip of the medical device from impinging on a lower surface of the cavity.

In some embodiments, a proximal portion of the medical device engages the opening in a luer lock engagement to provide a seal therebetween and prevent UV radiation escaping from the cavity.

In some embodiments, engaging a proximal portion of the medical device with the opening actuates the switching mechanism.

In some embodiments, the irradiation source is disposed between the outer surface of the housing and an inner surface of the cavity, the inner surface of the cavity is transparent or translucent, and the outer surface of the housing is opaque.

Also disclosed is a method of magnetizing and sterilizing a medical device including, placing a portion of a medical device into a cavity defined by a housing, the housing defining a top face, a bottom face and one or more side faces extending therebetween, the cavity disposed within the housing and communicating with an opening disposed in the top face, magnetizing the portion of the medical device using a magnetizing element disposed in a wall of the cavity, and sterilizing the portion of the medical device using an irradiation source disposed in a wall of the cavity and configured to direct ultraviolet radiation into the cavity.

In some embodiments, magnetizing the portion of the medical device includes imprinting a magnetic signature onto the medical device.

In some embodiments, the irradiation source is configured to emit electromagnetic radiation between the wavelength ranges of 100-280 nm.

In some embodiments, the irradiation source is configured to emit one or more of UV-A, UV-B, and UV-C radiation.

In some embodiments, the irradiation source is one of a LED bulb, a light bulb, an incandescent bulb, a fluorescent bulb, a fluorescent tube, or a low-pressure mercury-vapor gas-discharge lamp.

In some embodiments, the method further includes transitioning one or both of the magnetizing element and the irradiation source between an activated state and a deactivated state by a switching mechanism operably coupled with the opening.

In some embodiments, the method further includes closing a door or a flap over the opening to mitigate radiation escaping from the cavity.

In some embodiments, the medical device includes one of a needle, guidewire, or stylet that includes a magnetizable material.

In some embodiments, the method further includes engaging a proximal portion of the medical device with the opening to, i) align a distal portion of the medical device with a central axis of the cavity, ii) align the distal portion of the medical device at a predetermined distance from the irradiations source, or iii) prevent a distal tip of the medical device from impinging on a lower surface of the cavity.

In some embodiments, the method further includes engaging a proximal portion of the medical device with the opening in a luer lock engagement to provide a seal therebetween and prevent UV radiation escaping from the cavity.

In some embodiments, engaging a proximal portion of the medical device with the opening actuates the switching mechanism.

In some embodiments, the irradiation source is disposed between the outer surface of the housing and an inner surface of the cavity, the inner surface of the cavity is transparent or translucent, and the outer surface of the housing is opaque.

These and other features of the concepts provided herein will become more apparent to those of skill in the art in view of the accompanying drawings and following description, which disclose particular embodiments of such concepts in greater detail.

### BRIEF DESCRIPTION OF THE DRAWINGS

Embodiments of the disclosure are illustrated by way of example and not by way of limitation in the figures of the accompanying drawings, in which like references indicate similar elements and in which:

FIG. 1 illustrates a perspective view of a magnetizer system shown in cut-away, in accordance with embodiments disclosed herein.

FIG. 2 illustrates a front view of a magnetizer system shown in cut-away, in accordance with embodiments disclosed herein

FIG. 3 illustrates a top view of a magnetizer system, in accordance with embodiments disclosed herein.

FIG. 4 illustrates a front view of a magnetizer system shown in cut-away including a medical device, in accordance with embodiments disclosed herein.

FIG. 5 illustrates a top view of a magnetizer system including doors, in accordance with embodiments disclosed herein.

FIG. 6 illustrates a top view of a magnetizer system including a flap, in accordance with embodiments disclosed herein.

### DETAILED DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

In the following description, the terms “or” and “and/or” as used herein are to be interpreted as inclusive or meaning any one or any combination. As an example, “A, B or C” or “A, B and/or C” mean “any of the following, A, B, C, A and B, A and C, B and C, A, B and C.” An exception to this definition will occur only when a combination of elements, components, functions, steps or acts are in some way inherently mutually exclusive.

With respect to “proximal,” or a “proximal portion” of, for example, a medical device disclosed herein includes a portion of the medical device intended to be relatively nearer to a designated location. Likewise, a “proximal length” of, for example, the medical device includes a length of the medical device intended to be relatively nearer to or in the designated location. A “proximal end” of, for example, the medical device includes an end of the medical device intended to be relatively nearer to the designated location. The proximal portion, the proximal-end portion, or the proximal length of the medical device can include the proximal end of the medical device; however, the proximal portion, the proximal-end portion, or the proximal length of the catheter need not include the proximal end of the medical device. That is, unless context suggests otherwise, the proximal portion, the proximal-end portion, or the proximal length of the medical device is not a terminal portion or terminal length of the medical device.

With respect to “distal,” or a “distal portion” of, for example, a medical device disclosed herein includes a portion of the medical device intended to be relatively further from a designated location. Likewise, a “distal length” of, for example, the medical device includes a length of the medical device intended to be relatively further from the designated location. A “distal end” of, for example, the medical device includes an end of the medical device intended to be relatively further from the designated location. The distal portion, the distal-end portion, or the distal length of the medical device can include the distal end of the medical device; however, the distal portion, the distal-end portion, or the distal length of the medical device need not include the distal end of the medical device. That is, unless context suggests otherwise, the distal portion, the distal-end portion, or the distal length of the catheter is not a terminal portion or terminal length of the medical device.

To assist in the description of embodiments described herein, a horizontal plane is defined by the front to back axis and the left to right axis. A vertical plane extends normal to the horizontal plane.

Unless defined otherwise, all technical and scientific terms used herein have the same meaning as commonly understood by those of ordinary skill in the art.

### Magnetizer System

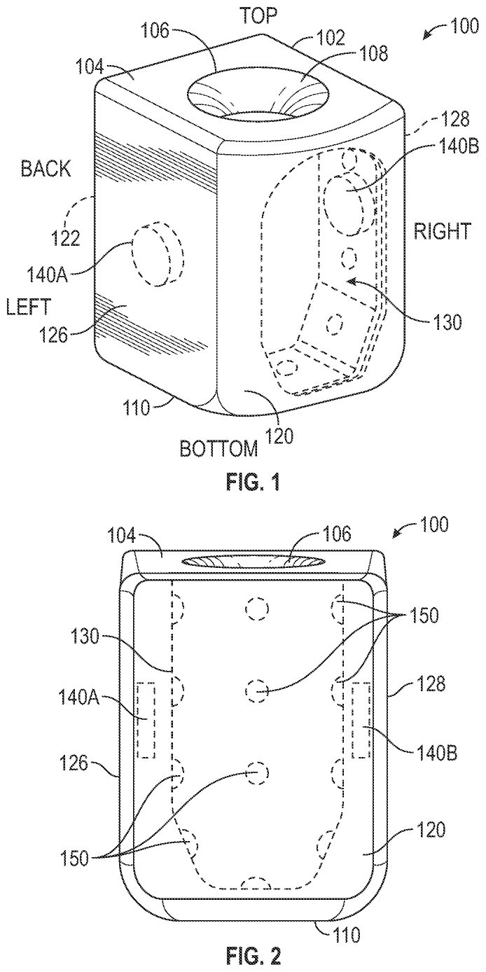

FIGS. 1-6 show embodiments of a magnetizer system (“system”) 100 for magnetizing a medical device 420 while maintaining the sterility of the medical device 420. Exemplary medical devices 420 include needles, guidewires, stylets, or similar elongate medical devices configured to be disposed within a body of a patient. In some embodiments, the magnetizer system 100 may be implemented with a magnetic-based guidance and tracking systems for insertion and detection of medical devices (e.g., a magnetic-based needle guidance system for insertion and detection of sterilized needles). Examples of magnetic-based device tracking and guidance systems are disclosed in U.S. Pat. Nos. 8,388,541; 8,781,555; 8,849,382; 9,456,766; 9,492,097; 9,521,961; 9,554,716; 9,636,031; 9,649,048; 10,449,330; 10,524,691; and 10,751,509, each of which is incorporated by reference in its entirety into this application.

In an embodiment, the medical device 420 includes a magnetizable material that enables the medical device (e.g., needle 420) to be magnetized, and which may then be tracked by a magnetic tracking system. Exemplary magnetic tracking systems include an ultrasound-imaging system that includes one or more magnetic sensors. When the magnetized medical device 420 is brought into proximity of magnetic sensors (e.g., a magnetic-sensor array) and/or inserted into the body of the patient during an ultrasound-based medical procedure, the magnetic tracking system differentiates between two or more of such magnetized medical devices and determines a location and orientation for each. Such magnetic-based tracking of the magnetized medical device assists a clinician in placing a tip of the medical device in a desired location, such as in a lumen of a blood vessel, by superimposing a simulated needle image representing the real-time location, distance and orientation of the needle over an ultrasound image of the body of the patient being accessed by the magnetized medical device.

In an embodiment, a magnetizable medical device 420 can be formed of stainless steel, however, other suitable magnetizable materials are contemplated to fall within the scope of the present invention. So configured, the magnetized needle 420 can produce a magnetic field or create a magnetic disturbance in a magnetic field detectable by the magnetic-sensor array of a magnetic tracking system (e.g. the ultrasound probe) so as to enable the location, distance and orientation of the magnetized medical device 420 to be tracked by the ultrasound-imaging and magnetic tracking system.

FIG. 1 shows a perspective view of the magnetizer system 100 shown in cut-away. FIG. 2 shows a front side view of the magnetizer system 100 shown in cut-away. The magnetizer system 100 generally includes a housing 102, a magnetization element 140 and an irradiation source 150, as described in more detail herein. The housing 102 comprises a top face 104, a bottom face 110, and one or more side faces extending therebetween, e.g. front face 120, back face, left side face 126, and right-side face 128. As shown the housing 102 defines a substantially cuboid shape, however it will be appreciated that this is exemplary and that the housing 102 can define other regular or irregular three-dimensional shapes such as cylinders, prisms, spheres, or the like, and include one or more flat, concave, or convex facets, and/or one or more rounded, chamfered, or beveled edges, without limitation.

In an embodiment, the housing 102 defines an interior cavity (“cavity”) 130. The cavity 130 communicates with an opening 106 disposed on a surface of the housing 102, for example the top face 104. The opening 106 further includes a curved portion 108 extending about a circumference of the opening 106 to define a chamfered or beveled entrance to the opening 106. The curved portion 108 is configured to facilitate directing a portion of the medical device 420 through the opening 106 and into the cavity 130, as described in more detail herein. In some embodiments, the opening 106 may be a hole, aperture, gap, slot or other opening formed by the top face 104 which permits a medical device 420 to enter and move therethrough. In an embodiment, the opening 106 defines a substantially circular aperture. However, this is not intended to be limiting and in embodiments, the opening 106 can define any closed curve, regular or irregular shape, such as a rectangle, oval, etc.

In some embodiments, the opening 106 is configured to facilitate user insertion and removal of a medical device (e.g., a needle) 420 to/from the cavity 130 of the magnetizer system 100. For example, the opening 106 may have a width suitable for enabling a user holding a needle 420 to briefly insert and remove the needle 420 in a dipping motion, thereby magnetizing the needle 420 within the magnetizer system 100. In an embodiment, the user may rest a medical device 420 on the opening 106 and/or the curved portion 108 so the medical device moves through the opening 106 in a controlled manner. In some embodiments, the curved portion 108 may comprise a fillet and/or a rounded portion. In further embodiments, the opening 106 may have proportions which are suitable for insertion of either a needle 420, or a needle 420 having a needle cover. The magnetizer system 100 may then be used even if the needle cover has not been removed from a needle 420.

In some embodiments, the opening 106, curved portion 108, cavity 130, or combinations thereof may have a length extending toward the bottom face 110 at least as long as the medical device 420. Such a length may reduce the chance that a medical device will contact the faces forming the cavity 130. In one embodiment, the user may magnetize the inserted medical device 420 by inserting the medical device 420 through the opening 106 until the medical device 420 contacts a bottom face 110 of the cavity 130, and then subsequently removes the medical device 420 through opening 106. However, the medical device need not contact the bottom face 110 of the cavity 130. In some embodiments, the top face 104, opening 106 and/or cavity 130 are least substantially free from structures which would impede the passage of a medical device 420 (e.g., needle) therethrough. As shown, the opening 106 is positioned in a center of the top face 104 to receive a medical device 420 for magnetization. However, as will be appreciated the opening 106 may be positioned in a corner of top face 104, on an edge of top face 104, or in other positions so that a medical device 420 may be received.

In an embodiment, the magnetizer system 100 further includes one or more magnetization elements 140 (e.g., permanent magnets and/or electro-magnets), which may be enclosed or encapsulated within a wall of the housing 102. For example, as shown in FIG. 1 a first magnetization element 140A is disposed between an outer left-side face 126 of the housing 102 and an interior left side face of the cavity 130. Similarly, a second magnetization element 140B is disposed between an outer right-side face 128 of the housing 102 and an interior right-side face of the cavity 130. As will be appreciated, the number, location, orientation, and configuration of the one or more magnetization elements 140 within the housing 102 is not intended to be limiting.

In some embodiments, the opening 106 may include structures to aid passage of a medical device 420 into the cavity 130 and near, or past, the magnetization elements 140. For example, in the embodiment of FIG. 1, opening 106 includes the curved portion 108 (e.g., a fillet). In some embodiments, opening 106 includes a curved, conical tapering surface, which extends from the top face 104 toward the cavity 130. For example, the opening 106 may be funnel-shaped, may taper or narrow, and/or may have a conical shape so that an inserted medical device 420 is directed toward the center of opening 106. In an embodiment, the opening 106/curved portion 108 may include one or more grooves configured to direct a portion of the medical device 420 towards the center of the opening 106. In an embodiment, a user may “dip” a medical device by placing the medical device on curved portion 108, causing the medical device to move toward the center of the opening 106, and then remove the medical device after it has been magnetized.

An opening 106 having a tapered shape may direct the movement of the medical device 420 to attempt to avoid contact between the medical device and other portions of the magnetizer system 100 (e.g., cavity 130). For example, in embodiments where a medical device to be magnetized is a needle, opening 106 may have a width which is equal to, or slightly larger than a width of the needle, so that the needle 420 may pass through. In some embodiments, the opening 106 is wider than the needle 420, to facilitate movement of the needle into and out of the magnetizer system 100. In some embodiments, a portion of the opening 106 may be narrower than a width of the cavity 130 so that a medical device 420 may be less likely to unintentionally contact an interior surface of the cavity 130.

In some embodiments, the opening 106 may be angled to accommodate a particular type of medical device. For example, opening 106 and/or the curved portion 108 may be straight (e.g., at least substantially perpendicular to the top face 104), or may be angled relative to the top face 104 with an angle of, for example, 1°, 1.72°, 10°, 15°, 30°, 45°, 60°, 75°, 88.28°, 89°, etc. In embodiments opening 106 and/or curved portion 108 may be include multiple angles, such as to accommodate a curved device.

In an embodiment, a distal portion of the medical device passes through the opening 106 and into the cavity 130 and a relatively proximal portion of the medical device is configured to engage the opening 106, e.g. curved portion 108. For example, a needle 420 is supported by a needle hub 410 at a proximal end. The needle 420 passes through the opening 106 and into the cavity 130, the needle hub 410 is configured to engage the opening 106 and/or curved portion 108 in a friction-fit, interference fit, luer lock, or snap-fit engagement.

Advantageously, the proximal portion, e.g. needle hub 410, engages the opening 106 and seals the distal portion, e.g. needle 420, within the cavity. Further, the hub 410 engaging the opening 106 aligns the needle 420 with a central vertical axis of the cavity 130. This places the needle 420 equidistant from, and/or at a predetermined distance relative to, one or both of the magnetization elements 140 and/or irradiation sources 150. Advantageously, the hub 410 and/or proximal portion of the needle 420 engaging the opening 106 prevents further movement into the cavity 130. As noted, the cavity defines a vertical length sufficient to receive the needle 420 therein. As such, the needle hub 410 engaging the opening 106 prevents the distal tip of the needle 420 impinging on a lower surface of the cavity 130 and prevents blunting of the needle 420 or damage to the cavity 130.

In an embodiment, the cavity 130 includes one or more irradiation sources 150 configured to emit ultraviolet (“UV”) radiation. Exemplary irradiation sources 150 can include LED bulbs, light bulbs, incandescent bulb, fluorescent bulbs, fluorescent tubes, low-pressure mercury-vapor gas-discharge lamp, or the like. In an embodiment, the irradiation sources 150 are configured to emit electromagnetic radiation between the wavelength ranges of 10-450 nm. In an embodiment, the irradiation sources 150 are configured to emit electromagnetic radiation between the wavelength ranges of 100-400 nm. In an embodiment, the irradiation sources 150 are configured to emit electromagnetic radiation between the wavelength ranges of 100-280 nm. In an embodiment, the irradiation sources 150 are configured to emit one or more of UV-A, UV-B, and UV-C radiation. Although it will be appreciated that other electro-magnetic ranges are also contemplated. In an embodiment, the irradiation sources 150 are configured to emit short-wave UV, germicidal UV, and/or ionizing radiation configured to sterilize a medical device 420 that is exposed to the radiation.

In an embodiment, the one or more irradiation sources 150 are disposed on a surface of the cavity 130 where one or both of the cavity 130 and housing 102 are formed of an opaque material that prevents any UV or optical light from passing therethrough. In an embodiment, the housing 102 (i.e. the outer faces thereof) are formed of an opaque material that prevents any UV or optical light from passing therethrough, and the interior walls of the cavity 130 are formed of a transparent or a translucent material. Further, the one or more irradiation sources 150 are disposed between the opaque walls of the housing 102 and the transparent/translucent walls of the cavity 130 and configured to direct the irradiation towards a central axis of the cavity 130. Advantageously, with the irradiation sources disposed between the wall of the cavity 130 and the wall of the housing 102, the surfaces of the housing 102 and/or cavity 130 can be wiped clean using cleaning solutions while protecting the irradiation source 150, and magnetization source 140, from exposure to the cleaning solutions.

As will be appreciated the number, location, orientation, and configuration of the irradiation sources 150 within the magnetizer system 100 can vary and are not intended to be limiting. In an embodiment, the one or more irradiation sources 150 emit electromagnetic radiation into the cavity 130 and onto at least a portion of the medical device 420 disposed within the cavity 130. As will be appreciated the number, location, and orientation of the one or more irradiation sources 150 are configured to emit sterilizing UV radiation on to a surface of the medical device 420 disposed within the cavity 130 to sterilize the medical device 420.

Advantageously, the magnetizer system 100 can simultaneously magnetize the medical device 420, to imprint a magnet signature thereon, while sterilizing or maintaining the sterility of both the medical device 420 and the cavity 130. For example, in use or in storage, the magnetizer system 100 may accumulate contaminants on its surfaces. Sterilizing the cavity 130 prevents any contaminants from being transferred from the magnetizer system 100 to the medical device 420 when the medical device 420 is being magnetized. Further, sterilizing the cavity 130 maintains the sterility of the cavity 130 during use. In addition, should the sterility of the medical device 420 have been compromised while outside of the magnetizer system 100, the irradiation sources 150 can re-sterilize the medical device 420, prior to subsequent insertion into the patient. Advantageously, the irradiation sources 150 allow a user to contact the medical device 420 with a surface of the magnetizer system 100 (e.g. curved portion 108) to allow for ease of guiding the medical device 420 into the cavity 130 without compromising the sterility of the medical device 420.

In an embodiment, the irradiation sources 150 can be activated or deactivated, i.e. transition between an activated or deactivated state. In an embodiment, the irradiation sources 150 can remain activated while the magnetizer system 100, and/or magnetic tracking system is/are in use. In an embodiment, the irradiation sources 150 can be activated when a medical device enters the cavity 130. In an embodiment, the magnetizer system 100 (e.g. opening 106) includes a mechanical, optical, or acoustic switching mechanism configured to determine the presence or absence of the medical device 420 within the cavity 130, or the insertion or removal of the medical device 420 therefrom. The switching mechanism is then configured to activate one or both of the magnetization elements 140 and the irradiation sources 150 when the medical device 420 is placed within the cavity 130.

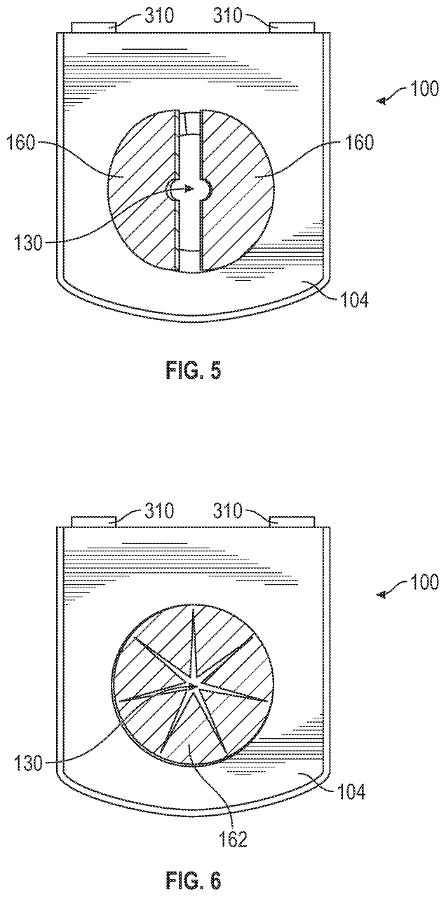

As shown in FIGS. 5-6, in an embodiment, the opening 106 may comprise a movable fixture configured to control access to the cavity 130. For example, as shown in FIG. 5, the opening 106 may include one or more doors 160 that are slidably or hingedly engaged with the magnetizer system 100 and which may be opened to allow access to the cavity 130, and closed to prevent access to the cavity 130. The one or more doors 160 can mitigate the introduction of contaminants to the cavity 130 in between uses. In an embodiment, the one or more doors 160 can be configured to close around a portion of the medical device 420 when the medical device 420 is engaged with the magnetizer system 100. In an embodiment, the one or more doors 160 can be operably coupled with the switching mechanism. As such, closing the one or more doors 160 can activate the one or more irradiation sources 150 and/or magnetization elements 140 as described herein. Advantageously, the one or more doors 160 can mitigate UV radiation from escaping from the cavity 130, mitigating exposure to a user.

In an embodiment, the magnetizer system 100 (e.g. opening 106) may comprise a flexible flap 162, cover or other barrier which mitigates contaminants from entering cavity 130 via the opening 106, and/or mitigates UV radiation from escaping from the cavity 130, while allowing ingress or egress of the medical device 420. For example, the flap 162 can be formed of a rubber, silicone rubber, or the like.

In an embodiment, a portion of the medical device 420, e.g. a proximal portion of the needle 420, and/or a needle hub 410, engages the opening 106 and/or curved portion 108 in a friction fit engagement as described herein. Advantageously, the needle hub 410 seals the needle 420 within the cavity preventing any UV radiation from escaping. In an embodiment, the needle hub 410 engaging the opening 106 transitions the switching mechanism between an activated state and a deactivated state.

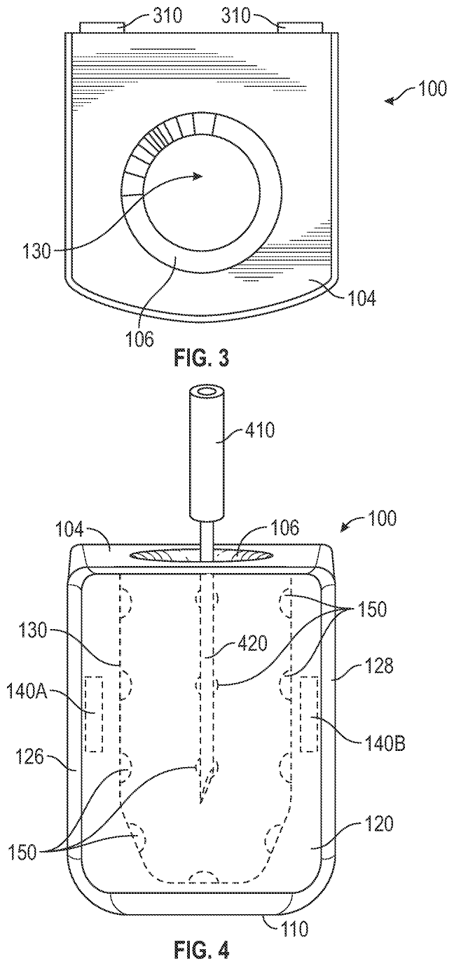

In an embodiment, as shown in FIG. 3, the magnetizer system 100 can include a brace 310 configured to couple the magnetizer system 100 to a surface, e.g. a surface a tracking system, ultrasound system, or the like. In some embodiments, the magnetizer system 100 may be attached to a clip, where the brace 310 may slide over or through a clip that is mounted to an ultrasound console. In such embodiments, a portion of the brace 310 may abut the ultrasound console. In an embodiment, the brace 310 can include a magnetic, adhesive, snap-fit, clip, latch, keyhole socket, or similar mechanism configured for securing the magnetizer system 100 to a surface, e.g. to a surface of the magnetic tracking system and/or ultrasound system.

### Ultrasound-Imaging Systems

In some embodiments, a magnetic tracking system includes an ultrasound imaging system. In some embodiments, the ultrasound-imaging system may include a console, a display screen, an ultrasound probe, and magnetizer system 100. An ultrasound-imaging system may be used to image a target such as a blood vessel or an organ within a body of a patient prior to a percutaneous puncture with a medical device 420 (e.g., needle) for inserting the needle into the target and accessing the target. Ultrasound-imaging systems may be used with a variety of ultrasound-based medical procedures, such as catheterization or to perform a biopsy of patient tissue. During use of the ultrasound imaging system, the clinician may need to reach out of a sterile field around the patient to control the ultrasound-imaging system. In some embodiments, the display screen may be used to display the distance and orientation of a magnetized medical device such as a needle. Distance and orientation data may be superimposed in real-time atop an ultrasound image of the target, thus enabling a clinician to accurately guide the magnetized medical device 420 to the intended target.

In some embodiments, the ultrasound probe is coupled to the console. The probe may be placed against a skin surface to generate ultrasound signals into a patient, receive reflected ultrasound signals or ultrasound echoes from the patient by way of reflection of the generated ultrasonic pulses by the body of the patient, and convert the reflected ultrasound signals into corresponding electrical signals for processing into ultrasound images by the console to which the ultrasound probe is communicatively coupled. In this way, a clinician can employ the ultrasound-imaging system to determine a suitable insertion site and establish vascular access with the needle or another medical device 420.

In some embodiments, an ultrasound probe can include a magnetic-sensor array for detecting a magnetized medical device 420 such as a needle during ultrasound-based medical procedures. The magnetic-sensor array includes a number of magnetic sensors embedded within or included on a housing of the ultrasound probe. The magnetic sensors are configured to detect a magnetic field or a disturbance in a magnetic field associated with the magnetized medical device when it is in proximity to the magnetic-sensor array. The magnetic sensors are also configured to convert the magnetic signals from the magnetized medical device (e.g., the needle) into electrical signals for the console to process into location, distance and orientation information for the magnetized medical device 420 with respect to the predefined target, as well as for display of an iconographic representation of the magnetized medical device on the display screen. Thus, the magnetic-sensor array enables the ultrasound-imaging system to track the needle or the like. The magnetic sensors may include three orthogonal sensor coils for enabling detection of a magnetic field in three spatial dimensions. In some embodiments, instead of 3-D sensors, a plurality of 1-dimensional (“1-D”) magnetic sensors can be included and arranged as desired to achieve 1-, 2-, or 3-D detection capability.

During operation of the ultrasound-imaging system, the ultrasound probe is placed against skin of the patient. An ultrasound beam is produced so as to ultrasonically image a portion of a target such as a blood vessel beneath a surface of the skin of the patient. The ultrasonic image of the blood vessel can be depicted and stabilized on the display screen of the ultrasound-imaging system.

The ultrasound-imaging system may be configured to detect the distance and orientation of a medical device, such as by way of the magnetic sensors. By way of example, the magnetic-sensor array of the ultrasound probe is configured to detect a magnetic field of the magnetized medical device or a disturbance in a magnetic field due to the magnetized magnetic device. Each magnetic sensor of the magnetic sensors in the magnetic-sensor array is configured to spatially detect the needle in 3-dimensional space. Thus, during operation of the ultrasound-imaging system, magnetic field strength data of the medical device's magnetic field sensed by each magnetic sensor of the magnetic sensors is forwarded to a processor of the console, which computes in real-time the distance and orientation of the magnetized medical device. The distance and orientation of the magnetized medical device is also for graphical display on the display screen.

The distance or orientation of any point along an entire length of the magnetized medical device in a coordinate space with respect to the magnetic-sensor array can be determined by the ultrasound-imaging system using the magnetic-field strength data sensed by the magnetic sensors. Moreover, a pitch and yaw of the needle 420 can also be determined. Suitable circuitry of the ultrasound probe, the console, or other components of the ultrasound-imaging system can provide the calculations necessary for such distance or orientation.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A magnetizer, comprising:
a housing defining a top face, a bottom face and one or more side faces extending therebetween;
a cavity disposed within the housing and communicating with an opening disposed in the top face, the opening and the cavity configured to receive a distal portion of a medical device therein;
a magnetizing element disposed in a wall of the cavity; and
an irradiation source disposed in the wall of the cavity and configured to direct ultraviolet radiation into the cavity.

2. The magnetizer according to claim 1, wherein the magnetizing element is configured to magnetize a portion of the medical device.

3. The magnetizer according to claim 1, wherein the magnetizing element is configured to imprint a magnetic signature onto the medical device.

4. The magnetizer according to claim 1, wherein the irradiation source is configured to emit electromagnetic radiation between a wavelength range of 100-280 nm.

5. The magnetizer according to claim 1, wherein the irradiation source is configured to emit one or more of UV-A, UV-B, and UV-C radiation.

6. The magnetizer according to claim 1, wherein the irradiation source is one of a LED bulb, a light bulb, an incandescent bulb, a fluorescent bulb, a fluorescent tube, or a low-pressure mercury-vapor gas-discharge lamp.

7. The magnetizer according to claim 1, wherein the opening further includes a switching mechanism configured to transition one or both of the magnetizing element and the irradiation source between an activated state and a deactivated state.

8. The magnetizer according to claim 1, wherein the opening further includes one of a door or a flap configured to mitigate radiation from the irradiation source escaping from the cavity.

9. The magnetizer according to claim 1, wherein the medical device includes one of a needle, guidewire, or stylet that includes a magnetizable material.

10. The magnetizer according to claim 1, wherein a proximal portion of the medical device engages the opening to:
i) align a distal portion of the medical device with a central axis of the cavity;
ii) align the distal portion of the medical device at a predetermined distance from the irradiation source; or
iii) prevent a distal tip of the medical device from impinging on a lower surface of the cavity.

11. The magnetizer according to claim 1, wherein a proximal portion of the medical device engages the opening in a luer lock engagement to provide a seal therebetween and prevent UV radiation escaping from the cavity.

12. The magnetizer according to claim 7, wherein engaging a proximal portion of the medical device with the opening actuates the switching mechanism.

13. The magnetizer according to claim 1, wherein the irradiation source is disposed between an outer surface of the housing and an inner surface of the cavity, the inner surface of the cavity is transparent or translucent, and the outer surface of the housing is opaque.

14. A method of magnetizing and sterilizing a medical device, comprising:
placing a portion of a medical device into a cavity defined by a housing, the housing defining a top face, a bottom face and one or more side faces extending therebetween, the cavity disposed within the housing and communicating with an opening disposed in the top face;
magnetizing the portion of the medical device using a magnetizing element disposed in a wall of the cavity; and
sterilizing the portion of the medical device using an irradiation source disposed in a wall of the cavity and configured to direct ultraviolet radiation into the cavity.

15. The method according to claim 14, wherein magnetizing the portion of the medical device includes imprinting a magnetic signature onto the medical device.

16. The method according to claim 14, wherein the irradiation source is configured to emit electromagnetic radiation between a wavelength range of 100-280 nm.

17. The method according to claim 14, wherein the irradiation source is configured to emit one or more of UV-A, UV-B, and UV-C radiation.

18. The method according to claim 14, wherein the irradiation source is one of a LED bulb, a light bulb, an incandescent bulb, a fluorescent bulb, a fluorescent tube, or a low-pressure mercury-vapor gas-discharge lamp.

19. The method according to claim 14, further including transitioning one or both of the magnetizing element and the irradiation source between an activated state and a deactivated state by a switching mechanism operably coupled with the opening.

20. The method according to claim 14, further including closing a door or a flap over the opening to mitigate radiation escaping from the cavity.

21. The method according to claim 14, wherein the medical device includes one of a needle, guidewire, or stylet that includes a magnetizable material.

22. The method according to claim 14, further including engaging a proximal portion of the medical device with the opening to:
i) align a distal portion of the medical device with a central axis of the cavity;
ii) align the distal portion of the medical device at a predetermined distance from the irradiation source; or
iii) prevent a distal tip of the medical device from impinging on a lower surface of the cavity.

23. The method according to claim 14, further including engaging a proximal portion of the medical device with the opening in a luer lock engagement to provide a seal therebetween and prevent UV radiation escaping from the cavity.

24. The method according to claim 19, wherein engaging a proximal portion of the medical device with the opening actuates the switching mechanism.

25. The method according to claim 14, wherein the irradiation source is disposed between an outer surface of the housing and an inner surface of the cavity, the inner surface of the cavity is transparent or translucent, and the outer surface of the housing is opaque.
